(19) United States
(12) Patent Application Publication
(10) Pub. No.: US 2020/0090814 A1
(43) Pub. Date: Mar. 19, 2020

(54) PROCESS, COMPUTER-ACCESSIBLE MEDIUM AND SYSTEM FOR GENERATING A BIOLOGICAL MODEL ASSOCIATED WITH DISEASE

(71) Applicants: Loes Olde LOOHUIS, New York, NY (US); BHUBANESWAR MISHRA, Great Neck, NY (US); ANDREAS WITZEL, New York, NY (US)

(72) Inventors: Loes Olde LOOHUIS, New York, NY (US); BHUBANESWAR MISHRA, Great Neck, NY (US); ANDREAS WITZEL, New York, NY (US)

(73) Assignee: New York University, New York, NY (US)

(21) Appl. No.: 16/689,727

(22) Filed: Nov. 20, 2019

Related U.S. Application Data
(63) Continuation of application No. 13/420,145, filed on Mar. 14, 2012, now abandoned.
(60) Provisional application No. 61/452,496, filed on Mar. 14, 2011.

(51) Int. Cl. G16H 50/50; G16B 5/00

(52) U.S. Cl. CPC G16H 50/50; G16B 5/00

(57) ABSTRACT

The present disclosure relates to exemplary embodiments of method, computer-accessible medium, system and software arrangements for, e.g., Cancer Hallmark Automata, a formalism to model the progression of cancers through discrete phenotypes (so-called hallmarks). The precise computational model described herein includes the automatic verification of progression models (e.g., consistency, causal connections, etc.), classification of unreachable or unstable states (e.g., “anti-hallmarks”) and computer-generated (individualized or universal) therapy plans. Exemplary embodiments abstractly model transition timings between hallmarks as well as the effects of drugs and clinical tests, and thus allows formalization of temporal statements about the progression as well as notions of timed therapies. Certain exemplary models discussed herein can be based on hybrid automata (e.g., with multiple clocks), for which relevant verification and planning algorithms exist.

### CROSS-REFERENCE TO RELATED APPLICATION(S)

This application claims priority from U.S. Provisional Patent Application No. 61/452,496, filed Mar. 14, 20011, which is hereby incorporated by reference in its entirety.

### FIELD OF THE DISCLOSURE

The present disclosure relates to translational systems biology, and in particular, to systems, processes, computer-accessible medium for obtaining a diagnosis, prognosis, evaluation, etc., for biomedical diseases and to exemplary biochemical systems which can be used for therapeutic intervention, for example.

### BACKGROUND INFORMATION

Cancer is generally thought of as a progressive disease—in particular, a disease which has certain discrete states through which it progresses towards a full-blown final phenotype (e.g., metastasis). This view is reflected in the so-called hallmarks of cancer (see Hanahan, D. and Weinberg, R. A., The hallmarks of cancer. Cell 100, 57-70 (2000)), and it has become one of the predominant ways of thinking about cancer, solidified through many further publications and experiments. A recent article (see Hanahan, D. and Weinberg, R. A., Hallmarks of cancer: The next generation. Cell 144, 646-674 (2011)) reviews and consolidates the new insights of the last decade.

According to such model, tumors can acquire certain “intermediate” hallmarks culminating in the “final” hallmarks of tissue invasion and metastasis. As provided in one such article,
Simply depicted, certain mutant genotypes confer selective advantage on subclones of cells, enabling their outgrowth and eventual dominance in a local tissue environment. Accordingly, multistep tumor progression can be portrayed as a succession of clonal expansions, each of which is triggered by the chance acquisition of an enabling mutant genotype. (Hanahan and Weinberg, 2011, p. 658)

The current list of cancer hallmarks includes the ability to reproduce autonomously, to ignore anti-growth signals, or to signal for formation of new blood vessels, as well as some other phenotypes. Hallmarks can be obtained in various different orders, but not every order may be viable. Intuitively, a hallmark can be acquired by a certain population of cells if it conveys a selective advantage compared to the predominant phenotype in that population. For example, in a wildly growing cluster of cells, the ability to signal for new blood supply, and thus nutrients, oxygen, and waste disposal, will allow the respective sub-population to outgrow the others. The notion of hallmarks can be further generalized to include other phenotypic equilibria and continuous progression between any two such generalized hallmarks can be measured and characterized by tumor growth curves, circulating tumor cells, heterogeneity, genomic instability, metabolism, blood supply to tumor, etc.

Most hallmarks can be acquired through mutations of very specific sets of genes, and many of the targeted drugs that have been developed in recent years influence the function of the products of these genes (Luo, J., Solimini, N. L., and Elledge, S. J., 2009. Principles of cancer therapy: Oncogene and non-oncogene addiction. Cell 136, 823-837). For example, the vascular endothelial growth factor (VEGF) signals for creation of new blood vessels (angiogenesis), and the drug Avastin inhibits the associated signaling pathway, thus preventing growing tumors from obtaining the needed blood supply.

The view of cancer progression and therapy can bear a close resemblance to formal models of state-transition machines in computer science. While cancer biologists organize their observations through these concepts, they do not have, or aim at, such formal models. Exemplary embodiments of the present disclosure include a formal framework called Cancer Hallmark Automaton (CHA) that can formally capture dynamics of cancer progression through accumulation of successive hallmarks governed by successive transitions.

The hallmark view of cancer, originally proposed by in the article by Hanahan and Weinberg (2000), and subsequently further modified by another article (Hanahan and Weinberg, 2011), includes the idea that carcinogenesis proceeds through a series of discrete phenotype states or hallmarks. Most hallmarks can be acquired through mutations of specific sets of genes, while global genomic instability drives the tumor progression through these hallmarks. These hallmark principles can also be highly relevant to the development of targeted therapies. See Table 1 for a small exemplary sample of therapeutic agents that attack specific hallmarks based on the product of the genes that they influence (Luo, et al., 2009).

Typically, these therapeutic agents are thought to act on the pathways that are behaving abnormally in the hallmark and thus restore normalcy (as in EGFR-mutated lung adenocarcinoma treated with Tarceva, or VEGF-mutated colorectal, lung, kidney, and glioblastoma cancers treated with Avastin). In these models, there may be no notion of time or history of the cancer progression.

Combinatorial approaches to cancer therapy can also help to improve treatment of the disease (Luo et al., 2009). For example, by combining several drugs affecting different mechanisms, or different signaling pathways used in a heterogeneous population, the progression to a next hallmark can be prolonged (or prevented).

Finally, many other parts of the tumor's host organism influence, or are influenced by, the tumor's progression and therapeutic agents: stroma, liver (see Rahman, A., Korzekwa, K. R., Grogan, J., Gonzalez, F. J., and Harris, J. W., 1994. Selective biotransformation of taxol to 6α-hydroxytaxol by human cytochrome p. 450 2c8. Cancer research 54, 5543), immune system (see de Visser, K. E., Eichten, A., and Coussens, L. M., 2006. Paradoxical roles of the immune system during cancer development. Nature Reviews Cancer 6, 24-37), stem cells, etc. Exemplary embodiments of the present disclosure can factor these parts of the host organism into the exemplary framework by modeling them separately and creating a suitable product automaton. It can then be possible to model the effects of a therapy on the whole system, describe interactions between subsystems and specify therapeutic goals over all of them. To illustrate such a composite model, we formulate a liver automaton which progresses through different states of damage depending on the toxicity of the given drugs, and show how this can be combined with a CHA (“Cancer Hallmark Automata”). The goal of a therapy is then to treat cancer without adversely affecting the liver. Other factors such as metabolic stress can also influence cancer progression, but can be incorporated into CHA components mutatis mutandis. (See, Jose, C., Bellance, N., and Rossignol, R., 2011. Choosing between glycolysis and oxidative phosphorylation: A tumor's dilemma? Biochimica et Biophysica Acta (BBA)—Bioenergetics 1807, 552-561; Luo, J., Solimini, N. L., and Elledge, S. J., 2009. Principles of cancer therapy: Oncogene and non-oncogene addiction. Cell 136, 823-837).

TABLE 1
Sample of therapeutic agents attacking specific
hallmarks. See Luo et al. at Table 1 (2009) (Providing
an extensive list and full references).

| Agent | Target | Hallmarks | References |
|---|---|---|---|
| ABT-737 | BCL-CL, BCL-2 | EvAp | Stauffer, 2007 |
| Alvocidib | CDKs | SSG | Lee and Sicinski, 2006 |
| Bevacizumab | VEGF | Ang | Folkman, 2007 |
| BEZ235 | PI3K | SSG, Ang | Maira et al., 2008 |
| GRN163L | hTERT | LRP | Dikmen et al., 2005; |
| | | | Harley, 2008 |
| Nutlin-3 | HDM2 | EvAp, IAG | Vassilev, 2007 |

Automata can represent formal frameworks to describe the (non-deterministic) behavior of discrete-state systems. These frameworks can range from simple finite automata, where states are described by nodes and transitions by edges, to complex state machines involving real-time progression and partial observability.

Timed Automata can extend classical automata to model progression of real-time systems. A timed automaton can include a finite automaton with a set of real-valued variables, called clocks. Clock constraints on the edges and clock invariants at the states can be used to restrict the possible progressions of the system.

Hybrid Automata can further extend timed automata to allow for non-synchronous continuous evolution. More precisely, while timed automata clocks increase synchronously at the same rate, clocks in so-called hybrid automata can run at different rates, which can change independently with the transition to another state.

Stochastic Automata can include stochastic state machines which satisfy the Markov property, e.g., their evolution only depends on the current state and not on the whole history of visited states. In that sense, they can also belong to the paradigm of automata. Markov models exist in a variety of forms. They can allow for partial observability (HMMs, Hidden Markov Models (see Rabiner, L. and Juang, B., 1986. An introduction to hidden markov models. ASSP Magazine, IEEE 3, 4-16)), for external control of the system's progression MDPs—Markov Decision Processes (see Puterman, M. L., 1994. Markov Decision Processes: Discrete Stochastic Dynamic Programming. John Wiley & Sons, Inc., New York, N.Y., USA, 1st edition.)) or both (POMDPs, Partially Observable MDPs (see Monahan, G. E., 1982. A survey of partially observable markov decision processes: Theory, models, and algorithms. Management Science 28, 1-16)).

System verification, and in particular model checking (see Clarke, E. M., Grumberg, O., and Peled, D. A., 1999. Model Checking. MIT Press), is concerned with formally verifying whether a given system satisfies a given property. Such a property could pertain to the (non-) reachability of certain good or bad states, or, more generally, be any temporal statement about visited states. Typically, a temporal logic like Computation Tree Logic (CTL, (see Clarke et al., 1986)) is used to express properties. There exist many extensions of CTL, e.g., Timed CTL. (See, Alur, R, Courcoubetis, C., and Dill, D., Model-checking in dense real-time. Information and Computation 104, 2-34 (1993)). It can facilitate statements not only about the qualitative temporal order of visited states, but also can include quantitative temporal operators. Timed CTL can be generalized further to reason about costs and the values of different clocks (see Bouyer, P., D'Souza, D., Madhusudan, P., and Petit, A., 2003. Timed control with partial observability. In Hunt, W. A. and Somenzi, F., eds., Computer Aided Verification, volume 2725, 180-192). System verification can become more difficult when the behavior of the system is not fully observable. In such situations of partial observability, the observer can narrow down the possible states of the system to a subset of states, but the exact state may not be known. In many formalisms that include partial observability, it is assumed that the observer is “automatically” notified whenever the currently available observation changes.

Control theory and planning can include the objective is to manipulate a system (e.g., “plant”) in such a way that the controlled system (“plant+controller”) satisfies a certain desired specification. Much work has been done on automatically generating controllers for untimed automata (see Jiang, S. and Kumar, R., 2006. Supervisory control of discrete event systems with CTL* temporal logic specifications. SIAM Journal on Control and Optimization 44, 2079-2103) for an algorithm that uses CTL specifications) as well as timed automata (see, e.g., Asarin, E., Maler, O., Pnueli, A., and Sifakis, J., 1998. Controller synthesis for timed automata. In Proc. IFAC Symposium on System Structure and Control, 469-474. Elsevier; Hoffmann and Wong-Toi, 1992). More recently, timed control with partial observability has received more attention (e.g., (Bouyer et al., 2003)). Cassez et al. (2007) show an example of an efficient on-the-fly controller synthesis algorithm for timed automata with partial information. (See, e.g., Cassez, F., David, A., Larsen, K. G., Lime, D., and Raskin, J., 2007. Timed control with observation based and stuttering invariant strategies. In Namjoshi, K. S., Yoneda, T., Higashino, T., and Okamura, Y., eds., Automated Technology for Verification and Analysis, volume 4762, 192-206). In hybrid automata theory, methods have been developed to design controllers for specific properties like non-reachability of bad states (so-called safety properties) as well as more general properties expressed in temporal logics, both using continuous and discrete-time control (see Henzinger and Kopke, 1999; Henzinger et al., 1999).

In the planning literature, algorithms have been developed to construct and validate action plans so that the resulting behaviors satisfy complex temporal formulas, called (temporally) extended goals. (See Bertoli, P., 2004. Planning with extended goals and partial observability. In Proceedings of ICAPS04, 270-278 planning under partial observability is studied, and in Quottrup, M., Bak, T., and Zamanabadi, R., 2004. Multi-robot planning: a timed automata approach. In Robotics and Automation, 2004. Proceedings. ICRA '04. 2004 IEEE International Conference, volume 5, 4417-4422 planning in real-time systems has been formalized. However, these methods have never been formalized and applied to such complex systems as CHA, alone or in combination with models for other organs.

Accordingly, there may be a need to address at least some of the above-described deficiencies.

### SUMMARY OF EXEMPLARY EMBODIMENTS OF THE DISCLOSURE

At least one of the objects of certain exemplary embodiments of the present disclosure can be to address the exemplary problems described herein above, and/or to overcome the exemplary deficiencies commonly associated with the prior art as, e.g., described herein. Accordingly, for example, described herein are exemplary embodiments of methods, procedures, computer-accessible medium and systems according to the present disclosure which can be used for obtaining diagnosis, prognosis, risk evaluation, therapeutic and/or preventive control based on generic or personalized models of various diseases, including cancer.

Exemplary embodiments of the present disclosure utilize a cancer hallmark automaton, which can be implemented as, e.g., a special case of a hybrid automaton. In the certain contexts that include an absence of reliable and sufficiently large amounts of data on expected outcomes from clinical tests, exemplary embodiments can implement nondeterministic models, which can capture uncertainty about state transitions, but may lack quantitative probability distributions. Thus, the resulting therapies can be highly conservative as they can allow planning against the worst-case behavior, rather than the average or expected case. Still, the exemplary framework can be justified by underlying stochastic processes involved in somatic evolution. Certain exemplary embodiments of the present disclosure, implementing a hybrid automata (as compared to a simple timed automata) can therefore be used for modeling how drugs can slow down those processes and thus affect their stopping times. Various drugs affect the transition to possible next states in different ways, and exemplary embodiments of the present disclosure can use multiple clocks to capture these processes.

Besides model-checking (e.g., verifying whether a certain set of properties is satisfied) these exemplary models of cancer progression, accordingly to certain exemplary embodiments of the present disclosure, it is possible to further utilize a therapeutic intervention against the progression of cancer—taking into account its partial observability. Exemplary notions of therapy can be defined consistent with various notions originally defined by control theorists in similar contexts. For example, a therapy can be defined as a function from the set of runs to the set of controller actions, e.g., administration of drug cocktails and medical tests. This exemplary utilization of the therapy can be translated into a conditional plan. Certain, exemplary embodiments of the present disclosure can use CTL and its extensions to specify therapeutic goals, and model partial observability. However, unlike conventional arrangements and methods for obtaining observations that are typically emitted automatically by the plant (e.g., any system being controlled), in exemplary embodiments of the present disclosure, it is possible to actively obtain the exemplary observations through test actions.

According to one exemplary embodiment of the present disclosure, system, process, method/process, and/or computer-accessible medium (e.g., including a hardware storage arrangement) can be provided in which, e.g., data related to the disease can be received; a temporal model and/or a spatio-temporal model of the disease can be created using the data; information associated with a set of properties related to a progression of the disease can be received; further information associated with at least one possible action for intervening with the progression of the disease can be obtained; and a model checking procedure can be performed for determining the properties and creating at least one counter-example if the model checking procedure results fail to meet a predetermined criteria. Further, it is possible to determine properties associated with the at least one of the disease, the progression of the disease or the at least one possible action, and the model checking procedure and creation of at least one counter-example can be repeated if the results fail to meet a predetermined criteria according to procedure. In addition, using a hardware processing arrangement, it is possible to modify the model using intervention steps to satisfy the properties. The data can be received from a source associated with omics, genomics and/or transcriptomics. It is also possible for the data to be received from a health record, which can be an electronic health record, and/or from a public information database. The exemplary model can be static, temporal and/or spatio-temporal.

Exemplary embodiments of the present disclosure can include methods, procedures, computer-accessible medium and systems which can be used for generating a biological model, which can include loading a plurality of states, each based on a state of a biological process; and determining a plurality of transitions between at least a subset of the plurality of states, wherein at least one transition includes at least one time-based attribute. The exemplary biological process can be a disease. The exemplary plurality of states can be based on data derived from at least one of clinical data and simulation data. The exemplary clinical data can be human clinical or animal clinical data. The exemplary plurality of states and the plurality of transitions can form at least one of a spatio-temporal model, a graph, an automata. The exemplary plurality of states and the exemplary plurality of transitions can form a hybrid automata wherein at least two transitions each includes at least one time-based attribute, and wherein the time-based attributes include clock rates that vary. The exemplary time-base attributes include clock constraints. The exemplary clock constraints include at least one of a maximum time value and a minimum time value.

Certain exemplary embodiments can include selecting at least one of a therapy and a treatment, based at least in part on the plurality of states, the plurality of transitions, and exceeding or meeting at least one goal. The exemplary goal can be at least one of a cost goal, a life-expectancy goal, a prevention of progression of a disease beyond a particular state goal, a disease progression prolongation goal, and an acceleration goal. The exemplary therapy and the exemplary treatment that can be selected include at least one of a drug, a surgery, and a radiation procedure. Certain exemplary embodiments can include selecting at least one test, based at least in part on the plurality of states and the plurality of transitions. The exemplary plurality of states can include partial observability. Certain exemplary embodiments can include selecting at least one of a therapy, a treatment, and a test, based at least in part on model checking. Further, the exemplary plurality of states and the exemplary plurality of transitions can define at least one model, wherein other models can be defined by other sets of states and transitions, and each model can be based on at least one of a disease, an organ, and a biological system. The at least one model and at least one of the further models can be combined into an overall model, which can have an associated overall goal. The overall model can be used to select a therapy or treatment to obtain the overall goal(s). Further, model checking can be used to verify properties of the biological process. Exemplary treatments can also be based at least in part on a synthesized controller, e.g., using control theory.

These and other objects, features and advantages of the present disclosure will become apparent upon reading the following detailed description of exemplary embodiments of the present disclosure, when taken in conjunction with the accompanying exemplary drawings and appended claims.

### BRIEF DESCRIPTION OF THE DRAWINGS

The foregoing and other objects of the present disclosure will be apparent upon Consideration of the following detailed description, taken in conjunction with the accompanying exemplary drawings and claims showing illustrative embodiments of the present disclosure, in which:

FIG. 1 is a block diagram of an exemplary hallmark automation in accordance with a first exemplary embodiment of the present disclosure;

FIG. 2 is a block diagram of the exemplary timed hallmark automation in accordance with a second exemplary embodiment of the present disclosure;

FIG. 3 is a block diagram of the exemplary hallmark automation with test observations in accordance with a third exemplary embodiment of the present disclosure;

FIG. 4 is an exemplary block diagram of an exemplary system in accordance with certain exemplary embodiments of the present disclosure;

FIG. 5 is a flow diagram of a method in accordance with certain exemplary embodiments of the present disclosure;

FIG. 6 is a block diagram of an exemplary anti-hallmark model using clocks x and y in accordance with certain exemplary embodiments of the present disclosure; and

FIG. 7 is a flow diagram of a method in accordance with certain exemplary embodiments of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the present disclosure and the appended claims.

### DETAILED DESCRIPTION OF EXEMPLARY EMBODIMENTS

According to exemplary embodiments of the present disclosure, it is possible to provide a formal framework called Cancer Hallmark Automaton (CHA) that can formally capture and/or utilize a cancer progression through an accumulation of successive hallmarks. States of these automata represent hallmarks, and directed edges among pairs of states can define paths, representing successive hallmark acquisitions.

Drugs can then be thought of as inhibiting specific transitions in a hallmark automaton. Systems, methods and computer-accessible medium according to certain exemplary embodiments of the present disclosure can leverage additional application of computer algorithmic techniques, namely controller synthesis, to aid in the increasingly complex task of designing therapeutic plans for cancer treatment. A therapy can be designed to satisfy a broad range of goals. For example, it is possible to have a preference for the system to stay within a specified region of the state space or satisfy a given temporal formula. Exemplary temporal formula goals can include goals called temporally extended goals. These exemplary goals can include an order in which certain states are reached, or a partial order in which certain states are reached (e.g., state X before state Y, etc.). These exemplary goals can be described using, e.g., temporal logic. The controller synthesis problem can then consist of finding a timed therapy that manipulates the model in such a way that the desired behavior is satisfied.

An exemplary hallmark view can model carcinogenesis abstractly as a progression through distinct phenotypes, following particular traces. Exemplary embodiments of the present disclosure can provide at least two advantages. On the one hand, it is possible to have the exemplary embodiments to facilitate practically all forms of cancers to be analyzed in one comprehensive and intuitive framework—without getting bogged down by their complexity. Additionally, according to exemplary embodiments of the present disclosure, it is possible to retain a sufficient level of detail to connect these phenotypes to specific genotypes and thus, to important low-level mechanisms involved in gene regulation, metabolism and signaling, some of which are accessible to various therapeutic agents.

Exemplary Hallmark models can utilize both of the above-described advantages to establish diagnostic categories, and to inform therapeutic decisions. By making all or various assumptions explicit and establishing a formal framework, exemplary embodiments can better explain the disease and its progression as well as its resilience against therapeutic interventions. For example, cocktail drugs are traditionally put together through trial and error, while the formal model of exemplary embodiments can help identify more rigorously exactly which (e.g., parallel or back-up) paths in cancer progression need to be perturbed/blocked by the cocktail. Certain exemplary embodiments can show, by including time in the formal framework explicitly, how cancer progression can be slowed down to the point that it is manageable as a chronic disease, rather than cured completely. Such exemplary approach can be preferable since it requires lower levels of toxicity.

Even though current literature in cancer biology typically lists no more than a dozen hallmarks, the resulting models are not necessarily simple or easy to analyze, and future progression models may make more course-grained or fine-grained reformulation of hallmarks. In addition, the list of targeted drugs has grown enormously, and the task of finding an optimal or near-optimal therapy plan may soon to be beyond traditional manual planning. An additional increase in complexity can result from combinatorial notions like synthetic lethality (Kaelin Jr, W. G., 2005. The concept of synthetic lethality in the context of anticancer therapy. Nat Rev Cancer 5, 689-698) and cocktail drugs, or path-dependent notions like oncogene-addiction (see Weinstein, I. B., 2002. Addiction to oncogenes-the achilles heal of cancer. Science 297, 63-64). Automated computational tools can help tackle these complexities. Model checking can be used to automatically verify hypotheses about cancer progression, and controller synthesis to generate suggestions for therapeutic plans, possibly personalized based on patients' genetics.

Starting from a rigorous formulation of cancer hallmark progression models, exemplary embodiments can implement a practical system. The model of exemplary embodiments can introduce a way for both the automatic generation of fine-grained hallmark models from data (e.g., The Cancer Genome Atlas project, see http://cancergenome.nih.gov) and their systematic usage in cancer diagnostics and therapeutics.

Model-discovery tools such as Gene-Ontology for Algorithmic Logic and Invariant Extraction (“GOALIE”) (Ramakrishnan, N., Tadepalli, S., Watson, L. T., Helm, R. F., Antoniotti, M., and Mishra, B., 2010. Reverse engineering dynamic temporal models of biological processes and their relationships. Proceedings of the National Academy of Sciences) can be used to generate models from data using any desired resolution for the state space. Ultimately, these models can be expected to be used for a wider variety of purposes, for example, mining clinical data to discover “bottlenecks” in cancer progression that point to promising drug targets, developing personalized models for specific cancers and patients, or even creating “expert systems” for clinicians and pathologists to query patient health records.

Exemplary embodiments of the present disclosure defines cancer hallmark automata, suggesting further extensions and providing some preliminary algorithmic considerations. The exemplary framework described in the present disclosure can start with, e.g., a simple Kripke model and adding the exemplary additional parameters:
(i) “costs” of drugs in various dimensions (e.g., toxicity, side effects, eventual resistance, mode and frequency of delivery, discomfort, monetary cost, etc.) that are to be optimized,
(ii) timing of transitions and drug effectiveness,
(iii) partial observability of the tumor's internal state along with tests that can provide additional information about the state, as well as
(iv) the possibility of factoring in other parts of the tumor's host organism which may be affected by a therapy (e.g., stroma, liver, immune system, stem cells, etc.).

According to certain exemplary embodiments of the present disclosure, it is possible to include a number of assumptions, and a CHA formalism. For example, an exemplary CHA can be modeled as a finite non-deterministic automaton whose nodes represent hallmarks and whose directed edges represent transitions from one hallmark to the next. The edges can be labeled with drugs that can inhibit the transition. A therapy can be defined as a function that assigns a set of drugs to each finite progression history, or run. An execution of a therapy can be defined as a run of the CHA that respects the therapy, that is, no transition of the execution is inhibited by the therapy. In certain exemplary embodiment of the present disclosure, it is possible to include costs by associating a cost vector with each state and each cocktail. Therapies can be selected by comparing costs of possible executions using a notion of Pareto dominance, in addition to the exemplary qualitative properties specified in CTL.

According to certain exemplary embodiments of the present disclosure, it is possible to extend the CHA framework to include real time. In this exemplary model, transitions can take certain durations of time, and drugs can prolong (or stop) the transition process. This can be modeled using a hybrid automaton with multiple clocks. Clock constraints on the edges and clock invariants at the states can restrict the possible progressions of the system. Multiple clocks can be used to allow for the scenario that a drug affects the transition to possible next states in different ways. Possible runs and therapies of a timed CHA can now include the clock values. An extension of CTL, Timed CTL, can be used to specify extended goals about the exemplary system.

According to certain exemplary embodiments of the present disclosure, it is possible to include uncertainty in the exemplary framework. The oncologist may have only partial knowledge about the tumor's internal state, which can be modeled by keeping track of his belief set. Tests can be incorporated into the definition of a therapy as actions that reduce uncertainty about the current state. In the exemplary framework, tests can have costs, but can take no time. To integrate the observer's information about the system, exemplary embodiments can add epistemic operators to Timed CTL. In further exemplary embodiments of the present disclosure, it is possible to provide a translation from therapies for timed CHAs with partial observability into conditional plans.

### Underlying Assumptions for Timed CHAs

There are some tacit assumptions that are used in certain exemplary embodiments and exemplary models (e.g., timed CHAs) and to a large degree, the structure of a therapy.
1. Exemplary models do not have to concern themselves with the origin of a cancer: e.g., no need to assume that cancer is a disease of the genome, initiated by a gain-of-function mutation in an oncogene or a loss-of-function mutation in a tumor suppressor gene, or a disease of aberrant signaling or a disease of addictive metabolic processes (e.g., Warburg effect), etc. It is possible to focus on the cancer phenotypes and their dynamics, without an explicit need for causal mechanistic models (which may be governed by genomics, epigenomics, transcriptomics, proteomics, mateabolomics, etc).
2. Exemplary models can postulate finitely many discrete phenotypes that can be exhibited in cancer. The dynamics, possible transitions from one phenotype to another, can be known, since they could be extracted by statistical analysis of patients, model animals, cancer cell lines or systems biology data. Exemplary models can be further extended to assume certain stress-hallmarks (e.g., certain characteristics of the tumor population, stroma or microenvironment) or other types of hallmarks.
3. Exemplary models' dynamics assumes that cancer progression is primarily driven by a Darwinian somatic evolution (e.g., based only on mutation and selection). In other words, exhibited changes in phenotypes can be determined by genotypic changes. Possible genotypic changes can be determined by various processes operating on the genome, e.g., point mutations, translocations, amplifications, deletions, loss of heterozygosity (LOH), which collectively may be labeled as a “Genome Organizing Device” (GOD). We only assume that GOD creates diversity, but not how exactly GOD functions. Advantageous cancer phenotypes of hallmarks are successively ‘selected’.
4. Exemplary models can further assume that each hallmark state has an associated (stochastic) hitting time, e.g., a particular instance of a stopping time, representing the first time the modeled process “hits” a successor hallmark, e.g., a well-defined subset of the state space. The stopping time can be modeled using Fisher's Fundamental Theorem of Genetics, and can be incorporated in the clocks of timed CHAs:

$$\frac{d}{dt} 〈 F 〉 = {σ}^{2} - μ {Δ}_{μ} .$$

where F represents a fitness function (corresponding to a hallmark) mapping genotypes to phenotypes, with the diversity in genotypes determining the variability in fitness, whose density function is over the population, F and σ2 are respectively the expectation and variance over the population and μ a mutation rate, and Δμ a contribution in average gain/loss in fitness due to mutation (which may be non-additive due to epistasis). A straight-forward derivation (with a simple model of genotypes), for the case involving mutation and selection (but no recombination) can be found in Neher, R. A. and Shraiman, B. I., 2011. Statistical Genetics and Evolution of Quantitative Traits. ArXiv e-prints.

The exemplary framework does not need to know how precisely the inter-hallmark clocks move, but only that they can be described by certain stochastic or ordinary differential equations, whose parameters are obtained from elsewhere. The fitness function can be assumed to change over time (e.g., an uncontrolled proliferative state may lead to hypoxia in a certain sub-population of cells and confer a higher fitness to mutations that promote angiogenesis, or other similar Malthusian effects), σ2 can be non-constant (e.g., the tumor's clonality and progression rates being variable), and also the mutation rates may vary over time. Thus, it is possible to obtain from the model, e.g., just the ability to represent the clocks mathematically, without being encumbered by the different mechanisms involved in different hallmarks.

Accordingly, these exemplary assumptions can facilitate a utilization of an exemplary model of cancer progression that can use a classical formalisms of hybrid automata with multiple clocks, whose mathematical and computational structures have been well-studied.

### Cancer Hallmark Automata

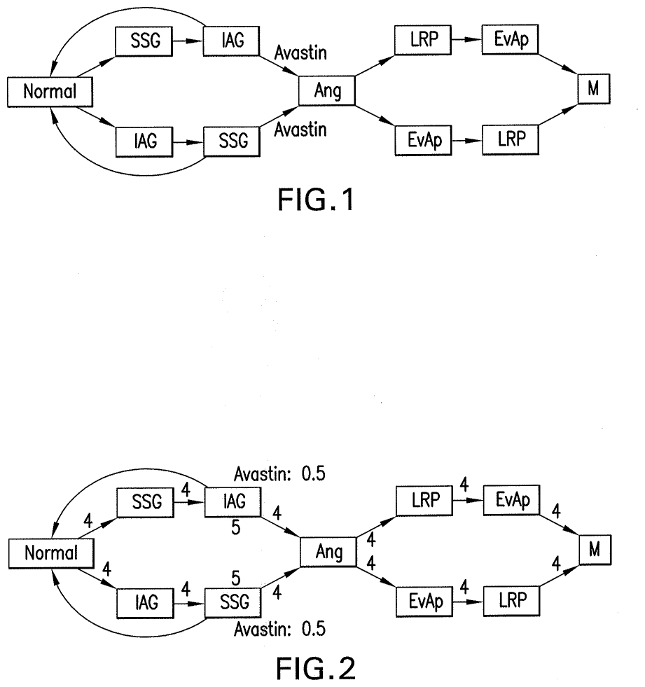

A block diagram of a simple, intuitive exemplary CHA according to an exemplary embodiment of the present disclosure is shown in FIG. 1. As illustrated in FIG. 1, this exemplary CHA includes the following hallmarks:
SSG: Self-sufficiency in growth signals. For example, cells may no longer depend on external growth-promoting signals, but grow autonomously. Usually, such state can be associated with a gain of function of an oncogene or a loss of function of a tumor suppressor gene.
IAG: Insensitivity to anti-growth signals. Cells with this hallmark can continue to grow even in the presence of inhibiting signals. Usually, certain cell-cycle checkpoints are no longer properly regulated.
Ang: Sustained angiogenesis. This state facilitates a cancer cell to signal for the construction of blood vessels.
LRP: Limitless replicative potential. While most normal cells can only divide a certain number of times, cells with this hallmark can divide without limits. In this state, a cancer cell may upregulate telomerase to restore telomere lengths.
EvAp: Evading apoptosis. Normally, cells have a program for controlled cell-death, which is used to remove damaged or otherwise unwanted cells. This program is disabled in this hallmark which enables cells with highly corrupted DNA to survive, which facilitates cancer.
M: Metastasis: Various possible progressions through these hallmarks can be seen as transitions in FIG. 1. For example, Ang can be acquired after SSG and IAG. Moreover, as mentioned in Section 1, if a growing tumor fails to acquire Ang, it may starve; in this case, a solid tumor is unable to grow further and attain the later hallmarks. For simplicity, it may be modeled as a transition to the normal state.

In the exemplary embodiment shown in FIG. 1, the exemplary therapy of, e.g., “give the drug Avastin whenever a state leading up to Ang is reached” will prevent the cancer from reaching M.

### Exemplary Formal Model/Procedure

In the following exemplary formal model, according to certain exemplary embodiments of the present disclosure, it is possible to start with a preliminary and simple formalization of the notions described above. It is then possible to successively extend the formal model into various exemplary implementations.

For example, an exemplary CHA can be defined as a tuple H=(V, E, v0), where V is a set of states, corresponding to hallmarks, E⊆V×2^{D}×V is a set of directed edges labeled with sets of drugs, and v₀∈V is the initial state. (Certain non-limiting examples described herein may omit the v0 and for simplicity, and simply state write (V, E).

An exemplary edge (v, D, v′) can represent a transition from state v to state v′ that can be inhibited by any drug from the set D⊆D. Exemplary embodiments can allow several drugs to be given simultaneously (or alternatively over short periods) and refer to such sets C⊆D of drugs as cocktails.

Given a cocktail C, the edge (v, D, v′)∈E can be inhibited by C if C∩D≠0. Given a state v and a cocktail C, v can transition to v′ under. C, in symbols v [symbol] v′, if there is an edge (v, D, v′) that is not inhibited by C. According to certain exemplary embodiments, it is possible to facilitate the use of multiple edges (e.g., with different labels) between two states. To prevent a transition between two states, e.g., most or all edges connecting them should be inhibited, which is why in certain exemplary embodiments, it is possible to consider cocktails rather than just single drugs. According to certain exemplary embodiments, it is possible to assume that for every state v and every cocktail C there exists some state v′ such that v [symbol] v′.

An exemplary run of a CHA H=(V, E, v0) can include a sequence of transitions in E. For example, Runs(v, H) can denote the set of runs that start in v. Runs(H) can denote Runs(v0,H), and Runsf(v,H) can denote the set of finite runs from Runs(v,H). In this context, an exemplary therapy can include a function π: Runs_{f}(H)→2^{D}. A possible execution of a in H can include a run:

S=v₀v₁v₂,
such that for each i≥0, v, [symbol] vᵢ₊₁, where Sᵢ denotes the initial segment of S up to step i.

Exemplary costs can be given by the following function, for some finite dimension n:
c: V→ [symbol] ⁿ≥0 Specifying costs of states,
c: 2^{D}→ [symbol] ⁿ≥0 Specifying costs of cocktails.

Thus, both exemplary states and exemplary cocktails can have costs assigned to them, represented as n-dimensional vectors. Dimensions can include toxicity of the drugs, monetary cost of the drugs, discomfort for the patients, etc. The exemplary cost of a possible execution S=v₀v₁v₂ . . . of a therapy π with discount factor 0<δ≤1 can include

$$c ( S , π , H ) = {\sum }_{i \geq 0} {δ}^{i} ( c ( {v}_{i} ) + σ ( π ( {S}_{i} ) ) ) .$$

An exemplary set of possible costs of a for a CHA H can include

c(π,H)={c(S,π,H)|S is possible execution of π in H}.

with these exemplary costs defined, e.g., the set of possible costs of a therapy, exemplary embodiments can compare different therapies with respect to their costs. Further exemplary cost definitions can include:
A cost vector x∈ [symbol] ⁿ Pareto-dominates another vector x′∈ [symbol] ⁿ, in symbols x [symbol] x′, if for each 1≤E≤n we have x_{f}≤x′_{f} and for some 1≤E≤n we have x_{f}<x′_{f}.

A therapy π Pareto-dominates a therapy π′ in a CHA H if for each x∈c(π,H) and x′∈c(π′,H) we have x [symbol] x′. The set of candidate therapies for H is

⊖(H)={π|π is not Pareto-dominated in H}.

For an exemplary case of 1-dimensional costs (or if there is a function to aggregate cost vectors into single numbers), the set of candidate therapies can be the set of therapies whose best-case cost is not higher than some other therapy's worst-case cost. This exemplary definition of a set of candidate therapies can be a very conservative one, in that it includes any therapy that is not overtly worse than some other therapy. Different possibilities can be provided for defining the set of candidate therapies, or for pruning the set further. Examples of such strategies for pruning the set further include miximin, e.g., choosing those strategies that lead to the best worst-case outcome, or maximax, e.g., choosing those strategies that lead to the best best-case outcome. However, making these decisions can depend on the risk attitude of patient and doctor which may not be fully formalizable. Therefore, in certain exemplary embodiments of the present disclosure, it is possible to include all the potentially relevant therapies in the set of candidate therapies.

In order to be clinically applicable, an exemplary hallmark model can be personalized for any given patient or cancer type. Richer exemplary models may utilize more personalization. This personalization can result in families of hallmark automata, with different sets of candidate therapies. In one example, for exemplary families of hallmark automata, a determination can be made as to whether there are any universal therapies for all of the included automata. Such therapies can result in faster and cheaper treatments. To be able to apply therapies across different automata, their domain may need to be the same. This can be achieved, for example, by considering CHAs that contain the same set of hallmarks, and therapies that either depend only on the current state, or that have the set of all sequences of states as the domain. The following exemplary definition can apply to therapies on such unified domains.

For example, given a family [symbol] of hallmark automata, the set of (universal) candidate therapies for [symbol] can include.

[symbol]

$$Θ ( ℋ ) = {⋂}_{H ∈ ℋ} Θ ( H ) .$$

A set θ of therapies covers [symbol] if

θ∩⊖(H)≠0 for all H∈H

Note that if ⊖(H)≠0 then for each π∈⊖(H), {π} covers H.

### Exemplary Temporally Extended Goals: CTL

As described herein, exemplary therapies can be compared according to their costs. Thus, the problem of finding the right therapy can be viewed as an optimization problem. It can, however, be beneficial to have more detailed control over the therapeutic objectives. Simple reachability properties can be used as goals, such as “metastasis will never be reached.” For more expressivity, certain exemplary embodiments can use Computation Tree Logic (CTL) (see Clarke and Emerson, 1982) to specify goals. (See, Clarke, E. and Emerson, E., 1982. Design and synthesis of synchronization skeletons using branching time temporal logic. In Kozen, D., ed., Logics of Programs, Lecture Notes in Computer Science, volume 131, 52-71).

The goal AG¬M can indicate that metastasis is never reached. Another possible goal can be

AG(Ang→AG¬EvAp)

This exemplary goal can mean that whenever sustained angiogenesis is acquired, then at no point in the future the capability of evading apoptosis will be obtained. According to certain exemplary embodiments of the present disclosure, it is possible to benefit from checking properties of the CHA itself, without application of a therapy. This can be done using CTL model checking (see, e.g., Clarke et al., 1999). CTL properties can also be checked on the possible executions of a given pair of therapy and hallmark automaton. Supervisory control for finite automata with CTL goals is known to be EXPTIME-complete, and controller synthesis algorithms exist (see Jiang and Kumar, 2006).

The above-described exemplary representation of a cancer hallmark automaton is intuitive, and simple. In addition, other exemplary embodiments according to the present disclosure can be used for added detail and complexity. For example, in further exemplary embodiments, it is possible to add timing and/or uncertainty about the current state. Typically, a clinician cannot know exactly how far cancer might have progressed, and may be forced to design therapies taking this uncertainty into account. Moreover, a clinician may decide to perform a test to reduce uncertainty. Additionally, cancer cannot be treated without considering the rest of the body. For example, it may be possible to prevent a cancer from evolving to metastasis but at the same time causing the liver to enter a highly toxic state. Thus, according to certain exemplary embodiments of the present disclosure, it is possible to include timed CHAs, for an exemplary robust model.

### Timed CHAs

In exemplary embodiments of the present disclosure, transitions can take certain durations of time, and drugs can slow down (or stop) the transition process. For example, in pancreatic cancer, it can take about a year for K-ras mutations in a cell to lead to neoplasms (so-called PanlNs) (see Hruban, R. H., Goggins, M., Parsons, J., and Kern, S. E., 2000. Progression model for pancreatic cancer. Clinical cancer research 6, 2969). According to certain exemplary embodiments of the present disclosure, it is possible to with the assumption that the acquisition of a hallmark requires a certain minimum amount of time. It is not necessary to specify exactly how that time is determined, but it can be the stopping time of a stochastic process such as randomizing over a set of driver mutations, or some value obtained from clinical data. After that time, e.g., a given transition will be possible, and as mentioned, drugs can be used to prolong this time.

Further, exemplary embodiments can allow states to have invariants, specifying the maximum time that the system can remain in the respective state. For example, a tumor may only be able to remain in a state of unbounded growth without angiogenesis for a certain number of months.

FIG. 2 shows a block diagram of the automaton from FIG. 1 with timing information added, illustrating this intuition according to a second exemplary embodiment of the present disclosure. FIG. 2 shows a block diagram of an exemplary timed hallmark automaton, using one clock (not named explicitly). The edges are labeled with the minimum times needed to make the respective transitions according to a second exemplary embodiment of the present disclosure. For example, in the two states that lead up to Angiogenesis, Avastin can be given to slow down the progress by a half. Those states are labeled with invariants, and depending on the precise timing, these invariants can force the system back to Normal before the transition to Angiogenesis is possible.

The exemplary extension can be formalized by assuming a finite set X of real-valued variables called clocks, over which the set of constraints C(X) is generated according to the grammar

ϕ::=x≥k|ϕ∧ϕ,

where k∈ [symbol] and x∈X. A valuation of the variables in X can include a mapping val: X→ [symbol] ≥0. Exemplary embodiments can denote a null valuation
x→0 by 0. By val |=ϕ the exemplary embodiments can denote that val satisfies ϕ. An exemplary timed CHA can be a tuple H=(V, E, v₀, I, p) where
V is a set of states, corresponding to hallmarks,
E⊆V×C(X)×V is a set of directed edges each labeled with a clock constraint,
vᵤ∈V is the initial state.
l: V×X→ [symbol] is a partial function specifying the time limit (if any) for each clock that the system can remain in a given state (this is also called the invariant), and
ρ: V×D×X→ [symbol] ≥0 yields a function specifying how a given drug influences the clucks at a given state.

Intuitively, at a given state v, the drug d can slow down or speed up the clock x as specified by the factor ρ(v, d, x). If the factor is 1 the drug has no effect on that clock, and if it is 0 it effectively stops the clock from progressing. If several drugs have an effect on a clock, their factors are multiplied. Thus, ρ can be extended to cocktails by setting ρ(v, C, x)=Π_{∂∈C}ρ(v, d, x) for any cocktail C≠0, and ρ(w, 0, X)=1

A directed edge (v, φ, v′) can represent a transition from v to v′ that can take place once the time constraint p is satisfied. We assume that for each state v that has a time limit for a clock x, there is an outgoing edge (v, φ, v′) such that val |=φ for all val with val(x)=f(v, x).

This edge can specify the behavior of the system if the respective clock reaches its time limit. The cost functions in the context of timed CHAs can be the same as those for the untimed version, but with a timed interpretation: c(v) is the cost of staying at state v per time unit (days/weeks/months/years), and c(C) is the cost of administering a drug cocktail C per time unit.

Next, according to certain exemplary embodiments of the present disclosure, it is possible to adapt the definitions related to runs of a CHA to the timed version, starting with the notion of a timed state. An exemplary timed state of a timed CHA (V, E) can be a tuple (v, val)∈V×R^{X}, where v is an exemplary hallmark state and val is an exemplary clock valuation. In certain exemplary embodiments, there can be two types of transitions between timed states:

1. Delay transitions, in symbols (v, val) [symbol] (v, val′), where
δ∈ [symbol] >0 represents the (real) time delay.
C denote the cocktail active during that time,
val′(x)=val(x)+δp(v,C,x) for all x, and
val′(x)≤l(v) for all x with l(v,x) defined.

2. State transitions, in symbols (v,val)→(v′,U), where
there it an edge (v,ϕ,n′)∈E with val |=ϕ

Whenever a state transition takes place, the clocks can be reset. This can simplify the presented examples, and could be replaced by explicit clock resets. This exemplary setup can include the special case where there is one clock unaffected by any drug, representing real time. Invariants over that clock can be used to specify, for example, how many months the tumor can remain in a certain hallmark state.

This exemplary timed setup can also emulate the concept of edges labeled with drugs that inhibit them. This can be achieved as follows: suppose certain exemplary embodiments were to model an edge between two states v, v′ that can be inhibited by a drug d. Then those exemplary embodiments can introduce a clock variable xd,v′ with ρ(v, d, xd,v′)=0, and add a constraint xd,v′≥z to the edge between v and v′, for some z>0. As long as drug d is given before the constraint is satisfied, the transition will be inhibited. However, once the constraint is satisfied, the tumor has advanced too far and it may no longer be possible to inhibit the transition.

An exemplary run in the case of a timed CHA H can be a non-Zeno sequence of delay and state transitions, that is, not containing an infinite chain of timed transitions with convergent total duration. Similar as before, let Runs ((v, val), H) denote the set of runs that start in (v, val). Further, write Runs (H) for the set Runs ((v0, 0), H), and Runs f ((v, val), H) for the set of finite runs from Runs ((v, val), H).

An exemplary therapy can include a function π: Runs_{f}(H)→2^{D}. A possible execution of π in H can include a run

S=(v₀,0)(v₁,val₁)(v₂,val₂)

suck that for all i with delay transitions (vᵢ, val;) [symbol] (vᵢ₊₁, valᵢ₊₁), for every 0≤δ′<δ

π((v_{O},0) . . . (vᵢ,val,)(vᵢ,valᵢ+δ′ρ(vᵢ,C)))=C,

where ρ(vᵢ,C) denotes the partial evaluation of ρ, i.e., the function x [symbol] ρ(vᵢ, C, x).

This last exemplary condition can ensure that the therapy does not change during a transition, or, put differently, that a change in therapy is always reflected by starting a new transition. For an exemplary finite run r∈Runs_{f}(H)· exemplary embodiments can denote its duration as

$$τ ( r ) = {\sum }_{\text{?}} \{ \begin{matrix}δ & if {r}_{j} {\to }^{δ , C} {r}_{j + 1} for some δ . C \\ 0 & otherwise\end{matrix} \\ \text{?} \text{indicates text missing or illegible when filed}$$

where len(r) denotes the length of the state sequence in r and rᵢ its initial segment of length i.

Given a CHA H and a possible execution S of a therapy π, the cost of S given π with discount factor 0<d≤1 can be expressed as:

$$c ( S , π , H ) = {\sum }_{i \geq 0} \frac{1}{d} ( {e}^{- {d}_{τ} ( {S}_{i} )} - {e}^{- d τ ( {S}_{i + 1} )} ) ( c ( {v}_{i} ) + c ( π ( {S}_{i} ) ) )$$

(as previously indicated, by Si the initial segment of S up to step i is denoted). This simple exemplary discounting function does not necessarily capture a real patient's preferences, but any exemplary convergent function will work in its stead.

The set of possible costs of π in a timed CHA H is the set of costs of possible executions of π,

c(π,H)={c(S,π,H)|S is possible execution of π in H}.

### Exemplary Timed CTL

According to certain exemplary embodiments of the present disclosure, it is possible to extend the CTL goals of the previous section to include time (Alur et al., 1993). For example, the goal AG≤20¬M says that metastasis is not reached within 20 time units (e.g., 20 years). This kind of goal can represent the approach of turning cancer into a chronic disease, rather than trying to cure it completely. For example, the above formula can be used for a patient of sixty years of age, who may then be able to obtain a less strenuous therapy, while for a younger patient the time requirements may be more extensive.

Out of all the therapies satisfying a CTL goal, the best ones may be chosen either by a separate cost optimization, or by incorporating cost requirements into the formulas using a weighted version of CTL (Bouyer, 2006).

### Exemplary Partial Observability and Tests

The previously-described exemplary embodiments of the present disclosure have assumed perfect information about the state of the system. Nonetheless, a clinician may only have partial observations of the tumor's internal state. To reduce uncertainty about the current state of the cancer progression, tests can be performed. According to certain exemplary embodiments (e.g., as discussed herein), it is possible to extend the previously discussed exemplary formal framework to include partial observability and tests, both for untimed and timed CHAs.

### Exemplary Tests in Untimed CHAs

According to certain exemplary embodiments of the present disclosure, it is possible to view tests as functions mapping states to observations. The exemplary test described herein can be deterministic, i.e., for any given state, a certain test can always lead to the same observation. Traditionally, non-deterministic tests are common, e.g., where a test may lead to one of a set of possible observations. The exemplary framework according to the present disclosure can be extended in the same or similar way, but from the biological perspective, which would mean that there are different mechanistic causes for the system being in that state. In that case, the exemplary model can be refined to have different states representing these different causes.

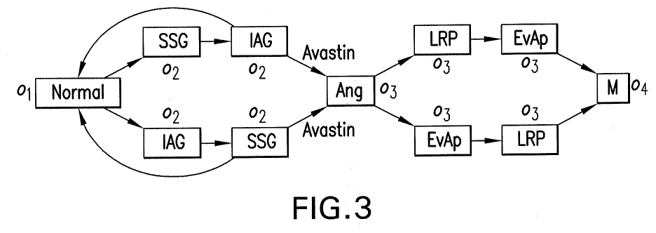

FIG. 3 shows an exemplary block diagram of such a test with 4 possible observations, according to a further exemplary embodiment of the present disclosure. When the test yields observation o2, the system is in a state prior to acquiring sustained angiogenesis, and that we can give a VEGF-inhibitor such as Avastin to inhibit the progression to a hallmark promoting construction of new blood vessels to the tumor. A more fine-grained test, or another test with intersecting observations, can be performed to determine the state more precisely, e.g., whether it is in the upper or in the lower branch of the automaton, and thus whether other potential drugs should be preferred.

Formally, for a CHA (V, E), according to certain exemplary embodiments, it is possible to assume a set T of tests and a set O of observations. Each test t′∈T can be a function t: V→O, inducing a partition on the set of states. When performing test t while the system is in state v, the resulting observation can facilitate the conclusion that the system is in one of the states in the equivalence class of v with respect to that partition. Thus, the exemplary framework and exemplary models can be extended to include tests. According to certain exemplary embodiments of the present disclosure, it is possible to assume that tests only acquire information, without affecting the state of the system. That is, given a test t and a state v, the exemplary system can only transition to v itself: v→t v.

According to certain exemplary embodiments of the present disclosure, it is possible to keep track of the information that results from tests by adding belief sets to runs. A belief set can be a subset of states that the system can be in at a given moment. States can be augmented with belief sets to obtain belief states.

An exemplary belief state of a CHA (V, E) can include a tuple (v, b), where
e∈V a state,
b⊆V with e∈b is belief set.⁰

There is a transitions from belief state (x,b) to (x′,b′) under a∈2^{D}∪T if

$$v {\to }^{a} {t}^{′} and$$

$${b}^{′} = \{ \begin{matrix}[ b ] \text{?} & if \text{?} b = C ∈ {2}^{D} \\ \{ {v}^{′} ∈ b \vert \text{?} = t ( c ) \text{?} \} & if a t ∈ T\end{matrix} \\ \text{?} \text{indicates text missing or illegible when filed}$$

where [X]_{R} denotes the image of set X under relation B, i.e., [X]_{R}={x′|(x,x′)∈R with x∈X}.

In symbols, it can be written as (v,b) [symbol] (v′,b′). In addition to an initial state v0, exemplary embodiments can now also define an initial belief set b0. So a CHA can now be a tuple (V, E, v0, b0), and a run of a CHA H is now a sequence of transitions over belief states. As described herein, runs ((v, b), H) can denote the set of runs that start in (v, b). Runs (H) can denote Runs ((v0, b0), H), and Runsf((v, b), H) can denote the set of finite runs from Runs ((v, b), H).

According to certain exemplary embodiments of the present disclosure, it is possible to extend the notions of therapies and their execution to include tests and belief sets. For example, a therapy can be a function π: Runs_{f}(H)→2^{D}∪T. The function can be uniform if it depends on the belief sets. An exemplary execution of π in H starting with (v0, b0) can include a run

S=(v₀,b₀)(v₁,b₁)(v₂,b₂) . . . ,

such that for each i≥0, (vᵢ, bᵢ) [symbol] (vᵢ₊₁, bᵢ₊₁).

According to certain exemplary embodiments of the present disclosure, it is possible to extend the definition of costs using c:T→ [symbol] ⁿ≥0 to specify costs of tests. The set of possible costs of π for a CHA H can include

c(π,H)={c(S,π,H)|S is a possible execution of π in H starting with (v,b₀) for any v∈b₀}.

### Exemplary Epistemic and Temporally Extended Goals

Given that the exemplary framework according to the present disclosure captures not only the actual behavior of the system but also the observer's (e.g., oncologist's) information about it, this additional aspect can be reflected in the formal language that defines goals. This can be done by adding an epistemic modality K to the logic, which can intuitively mean “it is known that.”

instead of the previously mentioned goal AG¬M, according to certain exemplary embodiments of the present disclosure, it is possible to express that it is known that metastasis is never reached by stating K AG¬M. Another goal can include:

AG(Ang→((¬M∧AX¬M)∪KAng))

which intuitively indicates that whenever the tumor acquires angiogenesis, this will be known (e.g., strictly) before the tumor reaches metastasis. Any such goal formula can implicitly be put inside an enclosing K operator to ensure that it holds in all starting states initially considered possible.

### Tests in Timed CHAs

Analogously to untimed CHAs, according to certain exemplary embodiments of the present disclosure, it is possible to extend the timed CHA framework to include belief sets and tests. A belief set b now is not just a set of states v, but a set of timed states (v, val). A belief state is a tuple (v, val, b) such that (v, val)∈b. As discussed herein, according to certain exemplary embodiments of the present disclosure, it is possible to assume some initial belief set b0 that is used when no other belief set is given.

In further exemplary embodiments, it is possible to introduce another relation. This exemplary relation can address the following issue: with full observability, individual delay or state transitions can be identified; however, with partial observability, a sequence of several transitions might look like just one transition to the outside observer. These multi-step transitions can be denoted using [symbol] , which can relate any two states that are related by any number of transitions under C taking a total time of δ. Formally, for two timed states (v, val) and (v′, val′), we have (v, val) [symbol] (v′, val′) if there exists a sequence S=(v,val)(v₁, val₁) . . . (v_{b}, val_{b})(v′, val′) of state or delay transitions under C, with τ(S)=δ. (Recall that τ(S) can denote the total duration of execution S.)

In exemplary timed CHAs with partial operability, there can be, e.g., at least three types of transitions between belief states:
1. D lag transitions, in symbols (v, val, b) [symbol] (v, val′, b′), where:
   (b, val) [symbol] (x, val′) and
   b′= [symbol]
2. State transitions, in symbols (x, val, b)→(v′, a, b′), where
   (v, val)→(v′, u) and
   b′= [symbol] , that is all state transitions under C
3. Test transitions, in symbols (x, val, b) [symbol] (v, val, b′), where
   b′={(i, v′, val′)∈b|v′∈t(r)}.

Tests in this exemplary formulation may only give information about the current state, and not about the current clock values. If deemed biologically plausible, this can be extended appropriately. Test transitions can be assumed to be instantaneous. This exemplary assumption can be made because receiving the result of a test can take hours or days, whereas tumors usually progress on a larger time scale (months or years).

An exemplary therapy can include a function π: Runs_{f}(H)→2^{D}∪T. An exemplary therapy can be uniform if it depends on the belief sets, and exemplary embodiments might only consider uniform therapies, without explicitly mentioning it. An exemplary execution of π in H can include a run

S=(r₀,0,b₀)(v₁,val₁,b₁)(v₂,val₂,b₂)

such that
for all i with delay transition (xᵢ, valᵢ, bᵢ) [symbol] (rᵢ₊₁, valᵢ₊₁, bᵢ₊₁) and for every u≤δ′, δ.

π((c₀,0,b₁₁) . . . (vᵢ,valᵢ,bᵢ)(vᵢ,valᵢ+δ′p(c₁,C), [symbol] ))=C,
where p(vᵢ, C) denotes the partial evaluation of p, i.e., the function x [symbol] p(vᵢ, C, x), and
for all i with test transition (rᵢ, valᵢ, bᵢ) [symbol] (vᵢ₊₁, valᵢ₊₁, bᵢ₊₁),

π((x′₀,a,b₀) . . . (rᵢ,valᵢ,bᵢ))=i.

### Exemplary Therapies as Conditional Plans

In this section, exemplary embodiments show how a therapy can be interpreted as a conditional plan instead of a function from runs to actions. Intuitively, a conditional therapy plan can include a sequence of therapeutic actions, which branches after each test action into distinct sub-cases according to the possible observations of the test. An exemplary formal translation of a therapy a into a conditional plan πc is given below. It may be noted that, due to uniformity, a therapy can be regarded as a function assigning actions to sequences of belief sets (rather than executions). bS can be the sequence of belief sets in S. When S is clear from the context, the subscript can be dropped and simply stated as b. By bob the sequence b is denoted with belief set b appended.

For example, given a sequence of belief sets b=b0 . : . bn, a time τ and a therapy π exemplary embodiments can define a conditional plan πc as follows:
If π(b)=C∈2^{D}, then

π_{c}(b,τ,π)=(C,τ):π_{c}(bo[bₐ] [symbol] ,τ+δ,π),
where δ is the minimum value such that
−π(bo[bₐ] [symbol] )≠C. and
−π(bo[bₐ] [symbol] )≠C for all δ′ such that 0␣δ′<δ. [symbol]
If π(b)=t∈T with possible observations o₁, . . . , oₖ, then

$$\text{?} ( b , τ , π ) = ( t , τ ) ; case [ \begin{matrix}\text{?} : \text{?} ( b ∘ ( \text{?} ⋂ {O}_{1} ) , τ , π ) \\ … \\ \text{?} : \text{?} ( b ∘ ( \text{?} ⋂ {O}_{k} ) , τ , π )\end{matrix} \\ \text{?} \text{indicates text missing or illegible when filed}$$

where Oᵢ={(b, val)∈V× [symbol] _{≥u}^{X}|t(v)=oᵢ}), and the case statement has the intuitive meaning, as explained below.

Given the initial belief set b0, the conditional plan that corresponds to the therapy π can be defined as πc (b₀, 0, π). Since a therapy may only depend on the sequence of belief sets, and the evolution of belief sets under any cocktail C can be predetermined, exemplary embodiments can compute when the therapy will change. For example, starting at the initial belief set b₀′ with initial cocktail C, the therapy changes at the smallest δ such that π( [symbol] )=C′ for some C′≠C. The new belief set at this moment can be b₁= [symbol] and the conditional plan up to this point can be (C,0); (C′,δ). The procedure can continue with the sequence b₀b₁. When a test is performed, the next move can depend on the observation oᵢ, which can be reflected in the branching case statement. The execution of such a therapy plan could then continue at the branch labeled with the observation.

### Exemplary Implementation: Liver and Product Automata

In a patient, cancer itself is not the only system of relevance. Other systems interact with the tumor's development, and especially during a therapeutic intervention, they may need to be monitored. For example, the immune system and its role throughout carcinogenesis are receiving more and more attention (de Visser et al., 2006), and the liver needs to be monitored to avoid damage due to excess toxicity.

In principle, other subsystems of an organism could be modeled as hybrid automata in the same way as our exemplary CHA, which could then be composed to an overall model for which therapies with goals spanning all subsystems could be generated. We postpone a discussion of the general framework and sketch here only a simple toxicity-based liver model that can be “attached” to a CHA. According to this exemplary embodiment, it is possible to use only one clock, modeling one type of toxicity level, and discrete dynamics governed by a sequence of thresholds.

A liver automation can include a tuple L=(W,F,w₁),l,ρ), where
W is a set of states.
F⊆W×W is a set of directed edges,
l:W→ [symbol] gives the toxicity threshold for each state, and
p:W×D→ [symbol] _{≥1} gives the toxicity factor for each pair of state and drug.

For simplicity, according to such exemplary embodiments of the present disclosure, it is possible to restrict attention to linear liver automata, e.g., each state can have at most one successor. For this reason, it is not necessary to provide explicit constraints on the edges, and instead assume that a state's outgoing edge is enabled exactly when its toxicity threshold is reached. The overall toxicity factor of a given cocktail in a given state can be defined as a function ρ:W×2^{D}→ [symbol] as follows:

$$ρ ( w , C ) = \{ \begin{matrix}{\prod }_{d ∈ C} ρ ( w , d ) & if C \neq ∅ \\ - 1 & if C = ∅\end{matrix}$$

For example, ρ(w,Ø)=−1, while for any C≠0, we have ρ(w,C)≥1. According to particular exemplary embodiments, it is possible to assume that drugs cumulatively increase the toxicity level, and that the liver regenerates only when no drugs are given. The exemplary model can easily be extended to include some drugs that have no effect on the liver, or to allow for other interactions between cocktails.

An exemplary timed state of a timed liver automation L=(W,F,w₀,l,ρ) can include a tuple (w, c), where m∈W is a current state and c∈R is a current clock value for w. In this exemplary embodiment, there can be, e.g., at least three types of transitions between timed states in the exemplary liver automation:
1. Detect transitions, in symbols (w,c) [symbol] (w,c′), where
   δ∈ [symbol] :−0 represents the (real) time delay,
   C denotes the cocktail active during that time,

$${c}^{′} = \{ \begin{matrix}max \{ 0 , c + δ p ( wC ) \} & if w = {w}_{0} , and \\ c + δ p ( w , C ) & otherwise\end{matrix} \\ - 1 \leq {c}^{′} \leq t ( ω ) .$$

2. State transitions, in symbols (w,r)→(w′,0), where
   c=l(w).
   (w,w′)∈F.
3. Regenerating transitions, in symbols (w,−1)→(w′,r′), where
   c′=0,
   (w′,w)∈F.

The exemplary thresholds for regenerating transitions can be modeled in more detail where required. A liver automaton can be combined with a CHA using parallel composition as usual in automata theory (see Henzinger, T. A., 1996. The theory of hybrid automata. In, Eleventh Annual IEEE Symposium on Logic in Computer Science, 1996. LICS '96. Proceedings, 278-292. IEEE). According to certain exemplary embodiments of the present disclosure, it is possible to then formulate combined goals about a CHA and the liver. For example, a goal might be to avoid a high level of toxicity (T5) in a somewhat advanced stage of the progression (Ang):

AG(Ang→¬T₅)

### Exemplary Procedures

According to certain exemplary embodiments of the present disclosure, it is possible to utilize procedures to define an exemplary formalism that captures the biology behind cancer progression models.

### From Timed CHAs to Hybrid Automata

Traditionally, in hybrid automata, the rates of the clocks are constant at any given state, and what is controllable are (some of) the transitions between states. In the exemplary framework, in contrast, the rates of the clocks is what can be affected by control actions (drugs), while the transitions (tumor progression) cannot be directly manipulated. However, this difference is mainly conceptual, and can translate CHAs to standard hybrid automata as follows, thus transferring existing results naturally.

Given a set of drugs D and a CHA with states V, exemplary embodiments can include a hybrid automaton H in the following way: for each state v∈V and each cocktail C∈2^{D}, H can contain a state vC with the same clock invariants as v. For any edge between two states v,v′∈V, H can contain an uncontrollable edge between vC and v′, for each cocktail C, with the same clock constraints and resets as on the CHA edge. In addition to the uncontrollable edges, there can be controllable directed edges from vC to vC′ for each v, C, and C′. These exemplary edges can represent changes of therapies, and can have no clock constraints or resets. At a state vC, the rate of each clock x∈X is fixed, given by ρ(v, C, x). The clock rates can be learned from patient data or mechanistically built on stochastic diffusion equations (SDEs) based on Fisher's theorem. This exemplary translation can yield a hybrid automaton of size exponential in the number of drugs, but linear in the number of CHA states.

### Exemplary Undecidability of Rectangular Hybrid Automata Control

It can be seen that the resulting exemplary hybrid automata can be related to an important subclass of “simple” hybrid automata called rectangular hybrid automata, whose clock-rates are defined using only upper and lower bounds. Rectangular hybrid automata can be an important subclass of hybrid automata because they can have nice computational properties (e.g., decidability). However, this complexity might only hold for those automata that satisfy initialization, meaning that whenever a transition changes the activity of a variable, the value of the variable is reinitialized. In fact, for rectangular hybrid automata without initialization, even the reachability problem can be undecidable.

### Exemplary Decidability of Discrete-Time CHA Control

One exemplary way around the undecidability of the hybrid automata control problem is to allow for control moves (e.g., therapeutic interventions) only at discrete instants of time. In one publication, an exponential-time algorithm is described for discrete-time safety control of rectangular hybrid automata with bounded and non-decreasing variables. (See, Henzinger, T. A. and Kopke, P. W., 1999. Discrete-time control for rectangular hybrid automata. Theoretical Computer Science 221, 369-392). The problem is shown to be EXPTIME-hard and discrete-time verification (CTL model checking) of rectangular hybrid automata to be solvable in PSPACE. Even though the exemplary definition according to certain exemplary embodiments can be timed CHAs does not require clocks to be bounded, such a restriction would not impose a severe limitation. By bounding the clocks by some value that even the healthiest patient will never reach, exemplary embodiments can thus aim for decidability without forfeiting any meaningful therapy.

### Exemplary Initialized Approximations of CHAs

Another exemplary method of ensuring decidability is by modeling the “belief automaton” explicitly, so that instead of controlling the underlying cancer progression model, control occurs only on the belief level. The modified CHA can be implemented by assuming that tests not only give information about the current state of the system, but also give some bound on how long the system has already been in this state. That is, at a given state, a test has a discrete number of possible outputs. Now, if it is required that every control action (e.g., change of cocktail) is preceded by a test action, all clocks can be set to the constants given by the test result, producing an exemplary initialized automaton. The resulting automaton can still be a rectangular hybrid automaton. In particular, the problem of safety control can become decidable.

### ADDITIONAL EXEMPLARY EMBODIMENTS

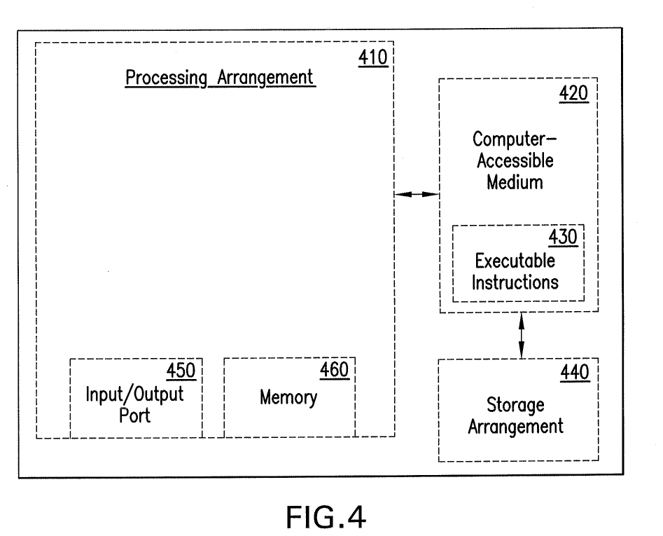

FIG. 4 shows an exemplary block diagram of an exemplary embodiment of a system according to the present disclosure. For example, an exemplary procedure in accordance with the present disclosure can be performed by a processing arrangement 410 and/or a computing arrangement 410. Such processing/computing arrangement 410 can be, e.g., entirely or a part of, or include, but not limited to, a computer/processor that can include, e.g., one or more microprocessors, and use instructions stored on a computer-accessible medium (e.g., RAM, ROM, hard drive, or other storage device).

As shown in FIG. 4, e.g., a computer-accessible medium 420 (e.g., as described herein, a storage device such as a hard disk, floppy disk, memory stick, CD-ROM, RAM, ROM, etc., or a collection thereof) can be provided (e.g., in communication with the processing arrangement 410). The computer-accessible medium 420 can contain executable instructions 130 thereon. In addition or alternatively, a storage arrangement 440 can be provided separately from the computer-accessible medium 420, which can provide the instructions to the processing arrangement 410 so as to configure the processing arrangement to execute certain exemplary procedures, processes and methods, as described herein, for example.

Further, the exemplary processing arrangement 410 can be provided with or include an input/output arrangement 450, which can include, e.g., a wired network, a wireless network, the internet, an intranet, a data collection probe, a sensor, etc. As shown in FIG. 4, the exemplary processing arrangement (computing arrangement) 410 can further be provided with and/or include exemplary memory 460, which can be, e.g., cache, RAM, ROM, Flash, etc. Further, the exemplary processing arrangement (computing arrangement) 410 can be in communication with an exemplary display arrangement which, according to certain exemplary embodiments of the present disclosure, can be a touch-screen configured for inputting information to the processing arrangement in addition to outputting information from the processing arrangement, for example. Further, the exemplary display and/or storage arrangement 140 can be used to display and/or store data in a user-accessible format and/or user-readable format. The exemplary processing/computing arrangement 410 shown in FIG. 4 can execute the exemplary procedures described herein, as well as those shown in the drawings.

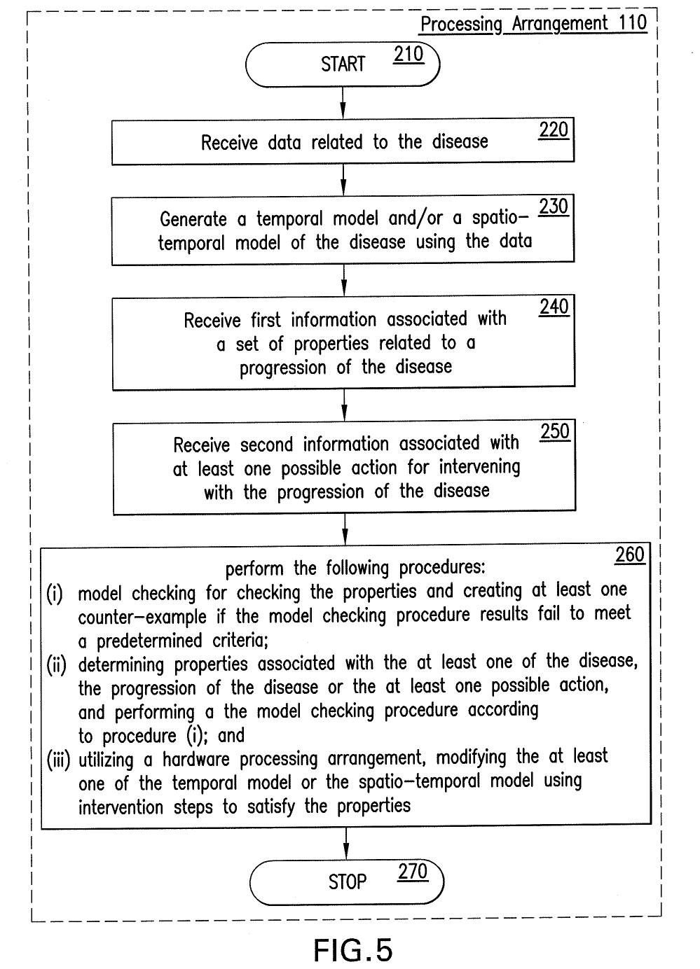

FIG. 5 illustrates an exemplary process/method showing exemplary procedures for generating information associated with at least one of diagnosis, prognosis, risk evaluation, therapeutic or preventive control of a progressive diseases such as cancer in accordance with certain exemplary embodiments of the present disclosure. As shown in FIG. 5, the exemplary procedure can be executed on and/or by, e.g., the processing/computing arrangement 410 of FIG. 4. For example, starting at subprocess 210, in accordance with certain exemplary embodiments of the present disclosure, the processing/computing arrangement 410 can, in subprocess 220, receive data related to the disease. In subprocess 230, the exemplary processing/computing arrangement 210 can generate at least one of a temporal model or a spatio-temporal model of the disease using the data. In subprocess 240, the exemplary processing/computing arrangement 210 can receive first information associated with a set of properties related to a progression of the disease. In subprocess 250, the exemplary processing/computing arrangement 210 can receive second information associated with at least one possible action for intervening with the progression of the disease. Then, in accordance with certain exemplary embodiments of the present disclosure, in subprocess 260, the exemplary processing/computing arrangement 210 can perform the following procedures: (i) model checking for checking the properties and creating at least one counter-example if the model checking procedure results fail to meet a predetermined criteria; (ii) determining properties associated with the at least one of the disease, the progression of the disease or the at least one possible action, and performing a the model checking procedure according to procedure (i); and (iii) modifying the at least one of the temporal model or the spatio-temporal model using intervention steps to satisfy the properties, for example.

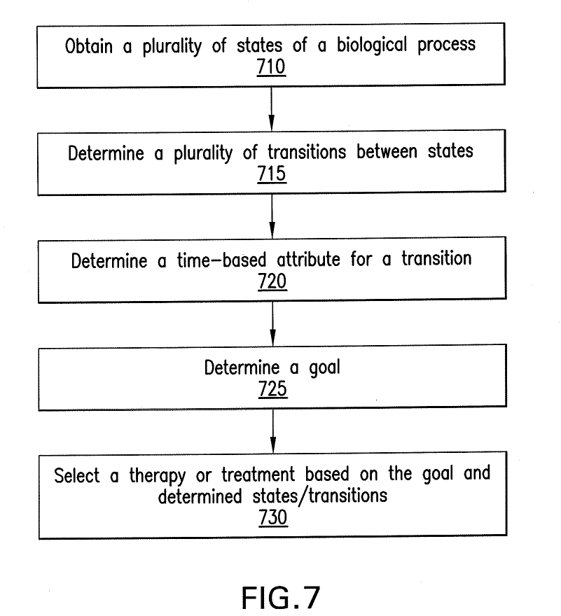

FIG. 7 illustrates another exemplary process/method showing exemplary procedures for generating a biological model. The exemplary procedure can start by obtaining a plurality of states representing a biological process at 710. Next, the exemplary procedure can determine a plurality of transitions between states at 715. This can include determining, loading, receiving, or otherwise establishing connections between the states. At least one of these transitions can have a time-based attribute, which can be determined or received at 720. The exemplary procedure can determine a goal at 725, which could be received from a user (e.g., doctor), a database, or any number of other sources. Finally, at 730, the exemplary procedure can select a therapy or treatment based on the states/transitions, and based on the goal, such as meeting or exceeding a minimum threshold. Optionally, exemplary procedures can perform model checking to verify properties of the disease progression. Optionally, exemplary procedures can use tools from control theory to select the therapy, treatment or a test.

Exemplary sets of states/transitions can comprise a model, and exemplary embodiments can include a database of several models. These models can be combined into an overall model, which can be used with other exemplary embodiments to, e.g., select a treatment/therapy.

One having ordinary skill in the art should appreciate that the teachings and/or disclosures described herein of exemplary embodiments of the present disclosure are not restricted to cancer. While certain exemplary embodiments provided and described herein have been in the context of cancer related diagnostics and treatments, exemplary embodiments of the present disclosure are applicable to other diseases, as well as other applications, including but not limited to, e.g., drug discovery and other properties, selection of drug and patient studies, explanation of risks and benefits of specific drugs and treatments, etc. Moreover, one having ordinary skill in the art should appreciate in view of the teachings herein that exemplary embodiments of method, computer-accessible medium and systems according to the present disclosure can be used for a large array of other applications, including applications in non-medical related fields, and will be equally applicable to similar problems to arise in the future.

### Modeling Heterogeneity and Anti-Hallmarks

Heterogeneous tumors: According to certain exemplary embodiments of the present disclosure, it is possible to provide modeled states of a CHA as representing the unique dominant phenotype of the tumor cell population. However, most forms of cancer are not likely to be monoclonal, e.g., consist of only one population in which the clonal expansions postulated by Hanahan and Weinberg take place, but rather involve several sub-populations of tumor cells (Navin, N., Kendall, J., Troge, J., Andrews, P., Rodgers, L., McIndoo, J., Cook, K., Stepansky, A., Levy, D., Esposito, D., Muthuswamy, L., Krasnitz, A., McCombie, W. R., Hicks, J., and Wigler, M., 2011. Trumour evolution inferred by single-cell sequencing. Nature 472, 90-94), each with a distinct dominant phenotype (see Fidler, I. J., 1978. Tumor heterogeneity and the biology of cancer invasion and metastasis. Cancer Research 38, 2651-2660; Heppner, G. H., 1984. Tumor heterogeneity. Cancer Research 44, 2259-2265. Hoffmann, G. and Wong-Toi, H., 1992. Symbolic synthesis of supervisory controllers. In American Control Conference, 1992, 2789-2793). In order to model this heterogeneity, exemplary embodiments can simply define a CHA state as representing a vector of dominant phenotypes, one for each sub-population. One or several components of such a vector may differ from one state to the next, corresponding to a change of the dominant phenotype in the Corresponding sub-population(s) during the respective transition; or the length of the vector may change, corresponding to new distinct sub-populations emerging or existing sub-populations dying out.

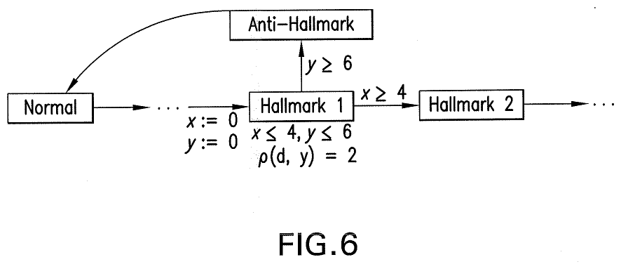

Anti-hallmarks: Instead of trying to slow down cancer progression, there has recently been growing interest in approaches to speed up the process to a degree which will make the tumor inviable and “push it over the edge” towards collapse. These inviable states can be referred to as anti-hallmarks. They can be modeled by putting constraints on the transitions leading to them that will never be satisfied, unless a drug is given which speeds up a certain clock. For example, consider the CHA in FIG. 6. At Hallmark 1, without interference (both clocks increase with rate 1), the transition to Hallmark 2 may occur after 4 time units. A drug that speeds up clock y by a factor of 2 will instead push the tumor to the Anti-Hallmark state, if given starting at most I time unit after entering Hallmark 1.

### CONCLUSION

The exemplary embodiments described herein establish, inter alia, a general formalism for describing cancer hallmarks and their dynamics, without necessarily relying on any detailed mechanistic model of cancer pathways (which could be included independently). The exemplary embodiments present a conceptually clear framework based on realistic biological foundations. While examples have been illustrated with respect to cancer, treatment, and modeling of the same, the exemplary framework can be used, as is, to model other exemplary phenomena.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements, and methods which, although not explicitly shown or described herein, embody the principles of the invention and are thus within the spirit and scope of the invention. In addition, all publications and references referred to herein are hereby incorporated herein by reference in their entireties. It should be understood that the exemplary procedures described herein can be stored on any computer-accessible medium, including, e.g., a hard drive, RAM, ROM, removable discs, CD-ROM, memory sticks, etc., included in, e.g., a stationary, mobile, cloud or virtual type of system, and executed by, e.g., a processing arrangement which can be or include one or more hardware processors, including, e.g., a microprocessor, mini, macro, mainframe, etc.

1. A non-transitory computer-accessible medium having stored thereon computer executable instructions for directing an application of at least one therapy for use in treating a cancer in at least one patient, wherein, when the executable instruction are executed by a computing hardware arrangement, configure the computing hardware arrangement to perform procedures comprising:
obtaining a plurality of states associated with the cancer based on a state of a biological process;
performing at least one test action on the at least one patient, wherein the at least one test action includes a therapeutic action from a therapeutic plan;
performing at least one control action on the at least one patient after performing the at least one test action, wherein the at least one control action includes at least one of at least one drug or at least one medical test;
establishing a plurality of transitions between at least one subset of the states, wherein at least one of the transitions includes at least one time-based attribute which includes multiple variable clock rates, each of the variable clock rates having its own respective constraint, and wherein each of the variable clock rates is based on (i) the at least one test action, and (ii) the at least one control action;
generating a hybrid automata model from the states and transitions;
determining the at least one therapy by comparing the hybrid automata model to a plurality of further hybrid automata models contained in at least one database; and
directing the application of the at least one therapy to treat the cancer in the at least one patient.

2. (canceled)

3. The computer-accessible medium of claim 1, wherein the states are based on data derived from at least one of clinical data or simulation data.

4. The computer-accessible medium of claim 3, wherein the clinical data is human clinical data or animal clinical data.

5. The computer-accessible medium of claim 1, wherein the states and the transitions form at least one of a spatio-temporal model, a graph, or an automata.

6-7. (canceled)

8. The computer-accessible medium of claim 1, wherein the clock constraints include at least one of a maximum time value or a minimum time value.

9. The computer-accessible medium of claim 1, wherein the computing arrangement is further configured to determine the at least one therapy based at least partially on the states and the transitions which exceed or meet at least one goal.

10. The computer-accessible medium of claim 9, wherein the at least one goal is at least one of a cost goal, a life-expectancy goal, a prevention of a progression of the cancer beyond a particular state goal, a cancer progression slowing goal, a temporally extended goal, or an acceleration goal.

11. The computer-accessible medium of claim 1, wherein the at least one determined therapy includes at least one of a further drug, a surgery, or a radiation procedure.

12. (canceled)

13. The computer-accessible medium of claim 1, wherein the states are partially observable.

14. The computer-accessible medium of claim 1, wherein the computing arrangement is further configured to determine the at least one therapy based at least partially on a controller synthesis procedure.

15. The computer-accessible medium of claim 1, wherein the further hybrid automata models are defined by further sets of further states and transitions, and wherein each of the further hybrid automata models is based on at least one of a disease, an organ, or a biological system.

16. The computer-accessible medium of claim 15, wherein the computing arrangement is further configured to combine the hybrid automata model and the further hybrid automata models into a combined model.

17. The computer-accessible medium of claim 1, wherein the computing arrangement is further configured to verify at least one property of the cancer based on at least one model checking procedure.

18. A method for treating a cancer in at least one patient, comprising:
obtaining a plurality of states associated with the cancer based on a state of a biological process;
performing at least one test action on the at least one patient, wherein the at least one test action includes a therapeutic action from a therapeutic plan;
performing at least one control action on the at least one patient after performing the at least one test action, wherein the at least one control action includes at least one of at least one drug or at least one medical test;
establishing a plurality of transitions between at least one subset of the states, wherein at least one of the transitions includes at least one time-based attribute which includes multiple variable clock rates, each of the variable clock rates having its own respective constraint, and wherein each of the variable clock rates is based on (i) the at least one test action, and (ii) the at least one control action;
generating a hybrid automata model from the states and transitions;
determining at least one therapy by comparing the hybrid automata model to a plurality of further hybrid automata models contained in at least one database;
applying the at least one therapy to the at least one patient.

19. A system for directing an application of at least one therapy for use in treating a cancer in at least one patient, comprising:
a computer hardware arrangement configured to perform procedures, comprising:
obtaining a plurality of states associated with the cancer based on a state of a biological process;
performing at least one test action on the at least one patient, wherein the at least one test action includes a therapeutic action from a therapeutic plan;
performing at least one control action on the at least one patient after performing the at least one test action, wherein the at least one control action includes at least one of at least one drug or at least one medical test;
establishing a plurality of transitions between at least one subset of the states, wherein at least one of the transitions includes at least one time-based attribute which includes multiple variable clock rates, each of the variable clock rates having its own respective constraint, and wherein each of the variable clock rates is based on (i) the at least one test action and (ii) the at least one control action;
generating a hybrid automata from the states and transitions;
determining the at least one therapy by comparing the hybrid automata model to a plurality of further hybrid automata models contained in at least one database; and
directing the application of the at least one therapy to treat the cancer in the at least one patient.

20. (canceled)

21. The computer-accessible medium according to 18, wherein the states are based on data derived from at least one of clinical data or simulation data.

22. The computer-accessible medium according to 18, wherein the data is received from (i) an electronic health record, or (ii) a source associated with at least one of omics, genomics or transcriptomics.

23. (canceled)
